(19) United States
(12) Patent Application Publication
(10) Pub. No.: US 2004/0102829 A1
(43) Pub. Date: May 27, 2004

(54) Multiple bend catheter for delivering a lead to a heart

(76) Inventors: Matthew D. Bonner, Plymouth, MN (US); Anthony Sze Leung Tang, Ottawa (CA)

Correspondence Address: Michael C. Soldner, Medtronic, Inc., MS 301, Mailstop LC340, 710 Medtronic Parkway, Minneapolis, MN, 55432, US

(21) Appl. No.: 10/302,289

(22) Filed: Nov. 22, 2002

(51) Int. Cl. A61N 1/05

(52) U.S. Cl. 607/125

(57) ABSTRACT

A catheter for introducing a lead into a heart that includes a hub and a shaft. The shaft forms a lumen that is adapted for positioning a lead therein, and extends from a proximal section coupled to the hub to a distal section including a distal end having a distal opening formed thereon. The shaft also includes an intermediate section positioned between the proximal section and the distal section. A proximal bend is formed along the shaft in a first plane, the proximal bend positioned between the proximal section and the intermediate section. A distal bend is formed along the shaft in a second plane, the distal bend positioned between the intermediate section and the distal section

### CROSS-REFERENCE TO RELATED APPLICATIONS

Cross-reference is hereby made to commonly assigned related U.S. application Ser. No. XX/XXX,XXX, filed concurrently herewith, to Terrell M. Williams, entitled “SYSTEM FOR COUPLING AN IMPLANTABLE MEDICAL DEVICE TO AN EPICARDIAL SITE” (Attorney Docket No. P-10411).

### FIELD OF THE INVENTION

The present invention is generally relates to implantable medical devices, and, more particularly, to a multiple bend catheter for delivery of a cardiac lead to a heart and various methods of placing heart leads in a human heart.

### DESCRIPTION OF THE RELATED ART

In pacemaker technology and related arts, a pacemaker and a pacing lead are implanted into a patient and coupled to a patient's heart to provide electrical stimulation of the heart such that the heart circulates blood in the desired fashion. The pacing leads may be introduced into the heart by a variety of techniques, and may be coupled to various areas of the heart. For example, in some cases, a transveneous lead, i.e., a lead positioned in a vein, is used to pace or apply other types of therapy to a heart.

Such transvenous leads may be positioned in a variety of cardiac veins, e.g., the great cardiac vein, the middle cardiac vein, the posterial lateral cardiac vein, the anterial lateral cardiac vein, or any other vein that drains into the coronary sinus of the heart. Such leads may also be placed in the right ventricle and right atrium, e.g., right ventricle pacing. In general, such leads are positioned in the vein by introducing the lead into the right atrium of the heart, inserting the lead through the coronary sinus ostium, positioning the lead in the coronary sinus, and directing the lead from the coronary sinus into the desired cardiac vein. The positioning that the lead follows may be very tortuous and may require negotiating sharp corners with the lead.

Typically, the placement of a lead in a cardiac vein is accomplished by use of a stylet/lead combination and/or use of a catheter. With respect to the first technique, a pacing lead, having a relatively stiff stylet positioned in a lumen formed in the lead, is inserted through the coronary sinus and into the desired vein. However, this process could, at times, be very difficult and tedious. That is, the introduction of a stylet-guided lead into a cardiac vein is basically a trial and error type of procedure in that the physician makes an estimation or guess as to the desired shape of the lead to properly access the desired vein. The stylet is bent to this estimated position and inserted into the lead. The stylet/lead combination is manipulated so as to access the coronary sinus and the appropriate cardiac vein. If the initial bend in the stylet does not enable the surgeon to access the desired vein, the lead may have to be withdrawn and the process repeated.

Another problem encountered in stylet delivered leads is that the lead may bend when pushed, i.e., the lead may lack sufficient rigidity. This may be problematic in some situations. For example, if the tip of a stylet-delivered lead is positioned in the coronary sinus, and a pushing force is exerted on the lead to push the tip further into the coronary sinus, the portion of the lead in the right atrium can bend into the right ventricle, thereby pulling the tip of the lead out of the coronary sinus. In such situations, a guide catheter may be used to keep the lead from bending in the right atrium.

In some prior art techniques, a single bend catheter may be employed in positioning a lead in a cardiac vein. That is, the distal end of such a catheter may be introduced into the coronary sinus. The single bend in the catheter is typically designed so as to make accessing the coronary sinus ostium and, ultimately, the coronary sinus itself, easier. Once the distal end of the catheter is positioned in the coronary sinus, a stylet-guided lead is inserted through the catheter and into the desired cardiac vein through the trial and error procedure generally discussed above. The primary benefit of having the catheter in the coronary sinus is to provide support for the lead portion crossing the atrium and minimizing friction between the lead and other tissue structures.

However, in both such techniques, introducing the lead into the desired cardiac vein is very difficult and tedious work due to several factors. For example, the typical stylet-guided lead experiences frictional and other impingement forces when an attempt is made to introduce the lead into the cardiac vein, i.e., when an attempt is made to “turn the corner.” Given the relatively flexible nature of the lead, the forces encountered in placing the lead into a cardiac vein may be difficult to overcome, or at least require a great deal of time and effort to do so.

Moreover, prior art techniques for positioning a lead in a cardiac vein often require performing an occlusion venogram to locate the desired cardiac vein. Such a process typically involves blocking the coronary sinus with, for example, a balloon-type catheter mechanism and injecting a fluoroscopic dye into the coronary sinus and the cardiac veins connected to the coronary sinus, i.e., the fluoroscopic dye was injected upstream through the coronary sinus. Using this technique, the surgeon could see the cardiac veins and select the desired vein for insertion of the lead. However, such dye, injected in such quantities, may be harmful to some patient's organs, such as a patient's kidneys. Thus, all other things being equal, it would be desirable to have a method of placing a lead in a cardiac vein in a more efficient manner, including in a manner that reduces the amount of fluoroscopic dye used in the procedure.

The present invention is directed to overcoming, or at least reducing the effects of, one or more of the problems described above.

### SUMMARY OF THE INVENTION

The present invention is generally directed to a uniquely shaped catheter that includes multiple bends. In one illustrative embodiment, a catheter for introducing a lead into a heart includes a hub and a shaft. The shaft forms a lumen that is adapted for positioning a lead therein, and extends from a proximal section coupled to the hub to a distal section including a distal end having a distal opening formed thereon. The shaft also includes an intermediate section positioned between the proximal section and the distal section. A proximal bend is formed along the shaft in a first plane, the proximal bend positioned between the proximal section and the intermediate section. A distal bend is formed along the shaft in a second plane, the distal bend positioned between the intermediate section and the distal section.

In another illustrative embodiment, a catheter for introducing a lead into a heart includes a hub and a shaft. The shaft forms a lumen adapted for positioning of the lead therein, and extends from a proximal section coupled to the hub to a distal section. The shaft includes an intermediate section positioned between the proximal section and the distal section, a proximal bend positioned between the proximal section and the intermediate section, and a distal bend positioned between the intermediate section and the distal section, with the proximal section, the proximal bend, and the intermediate section all positioned within a first plane. The distal section has a centerline that is oriented at a compound angle with respect to the first plane as defined by a vertical angle and a horizontal angle between the centerline of the distal section and the first plane.

In yet another illustrated embodiment, a catheter for introducing a lead into a heart includes a hub and a shaft. The shaft forms a lumen that is adapted for positioning a lead therein, and extends from a proximal section coupled to the hub to a distal section including a distal end having a distal opening formed thereon. The shaft also includes an intermediate section positioned between the proximal section and the distal section. A proximal bend is formed along the shaft in a first plane, the proximal bend positioned between the proximal section and the intermediate section. A distal bend is formed along the shaft in a second plane, the distal bend positioned between the intermediate section and the distal section. The second plane is positioned at an angle relative to the first plane, the proximal section and the intermediate section are positioned at a first angle in the first plane with respect to one another, the first angle ranging from approximately 90-165 degrees, the intermediate section and the distal section are positioned at a second angle in the second plane with respect to one another, the second angle ranging from approximately 75-150 degrees, the proximal bend has a radius of curvature of approximately 0.5-1.0 inches, the distal bend has a radius of curvature of approximately 0.25-0.75 inches, the proximal section has an axial length of approximately 20-40 inches, the intermediate section has an axial length of approximately 0.5-3.0 inches, and the distal section has an axial length of approximately 0.5-1.0 inches.

### BRIEF DESCRIPTION OF THE DRAWINGS

The invention may be understood by reference to the following description taken in conjunction with the accompanying drawings, in which like reference numerals identify like elements, and in which:

FIG. 1 is a perspective view of a catheter in accordance with an illustrative embodiment of the present invention;

FIG. 2 is a cross-sectional side view of a shaft of a catheter according to the present invention;

FIG. 3A is a side view of a catheter in accordance with an illustrative embodiment of the present invention;

FIG. 3B is an end view of the catheter of FIG. 3A;

FIG. 3C is a top view of the catheter of FIG. 3A; and

FIG. 3D is a side view of a distal bend of the catheter of FIG. 3C.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention will now be described with reference to the attached figures. The relative sizes of the various features and structures depicted in the drawings may be exaggerated or reduced as compared to the size of those features or structures on real-world devices. Moreover, for purposes of clarity, the devices depicted herein may not include all of the detailed components of a real-world implantable medical device. Nevertheless, the attached drawings are included to describe and explain illustrative examples of the present invention.

In general, the present invention is directed to a multiple bend catheter for delivery of leads to a human heart, and various methods of using same. Although various specific details are disclosed in describing the subject matter disclosed herein, the present invention should not be considered as limited to such details unless such details are specifically recited in the appended claims. Moreover, as will be understood by those skilled in the art after a complete reading of the present application, the present invention is not limited to the placement of cardiac pacing leads. Rather, the present invention may be used in placing any type of lead that provides electrical stimulation to, or senses electrical signals from, a patient's heart, e.g., a pacing lead, a defibrillation lead, a neurostimulation lead, and etc. Additionally, it should be understood that the catheter of the present invention may be used to introduce a lead into any cardiac vein that drains into the coronary sinus of the heart, e.g., the great cardiac vein, the middle cardiac vein, the posterial lateral cardiac vein, the anterial lateral cardiac vein, etc.

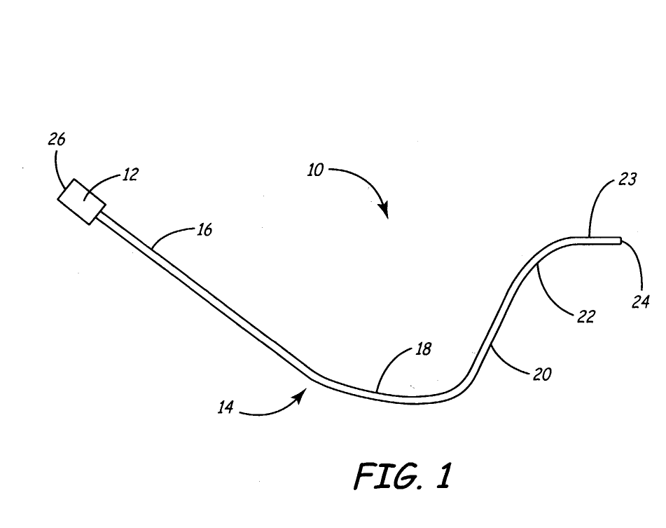

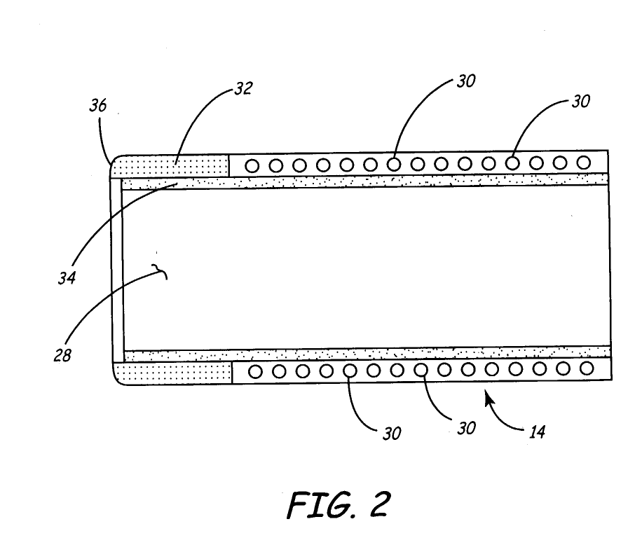

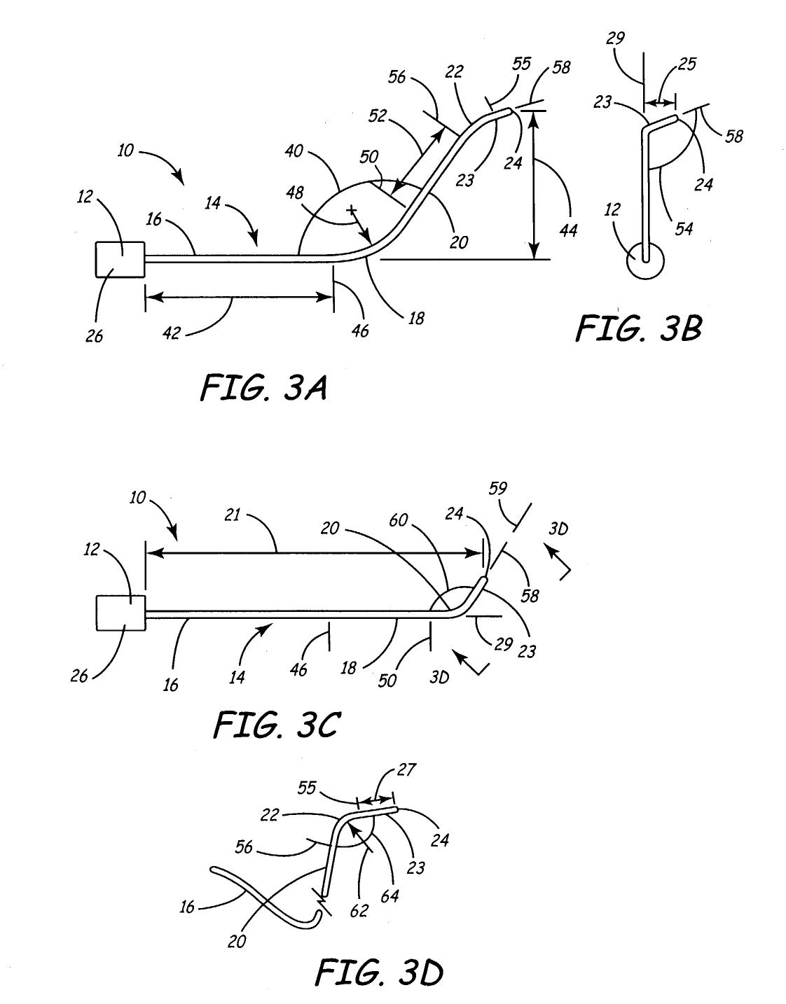

With reference to FIGS. 1 and 3A-3D, the catheter 10 is generally includes a hub 12 and a shaft 14 having a proximal section 16, a proximal bend 18, an intermediate section 20, a distal bend 22, a distal section 23, a distal opening 24, and a proximal opening 26. The shaft 14 also has a centrally located lumen 28 (see FIG. 2) that is adapted to receive a lead (not shown) therein. In general, the catheter 10 is designed and bent so as to enable efficient positioning of the distal opening 24 of the catheter 10 in a cardiac vein in which a lead is to be inserted. Thereafter, a lead is inserted within the proximal opening 26, through the lumen 28, and out the distal opening 24 into the selected vein in a manner to be described more fully below. The lead is then positioned and secured within the selected cardiac vein, and the catheter 10 is withdrawn from the patient.

The structure and composition of the shaft 14 may vary. FIG. 2 depicts one illustrative composition of the shaft 14, although others are possible. As shown therein, the shaft 14 may generally be formed of a polymer, such as PEBAX, and may have a hardness of approximately 75D. When the shaft 14 is manufactured, a material, such as barium sulfate, may be introduced into the shaft material such that the shaft 14 may be more opaque, thereby making the shaft 14 more easily observable during use.

In one embodiment, this material is only included within the catheter distal tip. The shaft 14 of the catheter may include a wire material 30 formed in the shaft 14. In one illustrative embodiment, shaft 14 includes braided metal that is a continuous pattern of 8×8 round medium tensile wires having a diameter of approximately 0.002 inch. Flat wires may be employed to minimize the catheter outer diameter. The different sections may have different reinforcing wires and structures, including coils, braids, polymers, mesh, etc. In some embodiments, the shaft 14 may further include a relatively soft tip 32 (less than approximately 40D) made of a polymer, such as PEBAX. The tip 32 may have an axial length of approximately 0.100 inches. The relatively soft tip 32 may not be used on all embodiments of the present invention. The shaft 14 may also include a polymer liner 34, such as PTFE, ETFE, etc. to increase the lubricity of the inner lumen. The corners 36 of the tip 32 may be rounded, and the liner 34 may be slightly recessed within the tip 32. The outer diameter of the shaft 14 may vary from approximately 4-10 French, and the wall thickness of the shaft 14 may be approximately 1 French. Of course, a lubricating coating may be used in lieu of the polymer liner 34. It should also be understood that the catheter 10 of the present invention may employ shafts 14 with a variety of different constructions. Thus, the present invention should not be considered as limited to the embodiments disclosed herein unless such limitations are clearly set forth in the appended claims.

The shaft 14 may be manufactured using traditional mandrel manufacturing techniques. That is, a mandrel (not shown) of the approximate desired finished shape of the catheter 10 may be provided. Thereafter, the liner 34 may be positioned over the mandrel, and the various components comprising the shaft 14, e.g., the braided wire material 30, may be positioned around the liner 34. Then, the shaft material, e.g., PEBAX, may be applied to this structure and heated and molded to result in the structure depicted in FIGS. 1 and 2.

In general, the present invention is directed to a uniquely shaped catheter 10 that is designed to aid in the rapid and accurate placement of a lead in a cardiac vein. This uniquely shaped catheter 10 is further depicted in FIGS. 3A-3D. FIG. 3A is a side view of the catheter 10. As shown therein, the proximal section 16 is separated from the intermediate section 20 by the proximal bend 18. Moreover, the centerlines of the proximal section 16 and intermediate section 20 are disposed at a vertical angle 40 relative to one another. In one illustrative embodiment, the angle 40 may range between approximately 90°-165°, in another example the angle 40 may range from approximately 125°-145°, and in another particularly illustrative embodiment, the angle 40 may be approximately 135°. The length 42 of the proximal section 16 may also vary from, for example, between approximately 20 and 40 inches. In one embodiment, the distal opening 24 is located above the centerline of the proximal section 16 by a distance 44 of approximately 1-3 inches. The proximal end 46 of the proximal bend 18 may be approximately 2-5 inches from the distal opening 24. The proximal bend 18 may have a radius of curvature 48 of approximately 0.5-1.0 inches.

The proximal end 50 of the intermediate section 20 may be approximately 1-3 inches from the distal opening 24, and the intermediate section 20 may have a length 52 that ranges from approximately 0.5-3.0 inches. As shown in FIG. 3A, the proximal end 56 of the distal bend 22 may be approximately 0.5-1.0 inches from the distal opening 24, and the distal end 55 of the distal bend 22 may be approximately 0.75-2.0 inches from the distal opening 24. The distal section 23 may have an axial length 27 (see FIG. 3D) of approximately 0.5-1.0 inches. As shown in FIGS. 3B and 3C, the centerline 58 of the distal opening 24 is positioned at a compound angle with respect to the plane 29 containing the proximal section 16, the proximal bend 18, and the intermediate section 20. This compound angle may be described by a vertical angle 54 (see FIG. 3B) and a horizontal angle 60 (see FIG. 3C). In one illustrative embodiment, the vertical angle 54 may be approximately 85°-105°, in another embodiment approximately 90°-100°, and in one particular embodiment approximately 98°. The distal opening 24 may be laterally offset from the centerline of the intermediate section 20 by a distance 25 of approximately 0.5-0.75 inches. In one illustrative embodiment, the horizontal angle 60 may vary being approximately 90°-150°, in another illustrative embodiment between approximately 100°-130°, and in one particular embodiment, the angle 60 may be approximately 110°. The shaft 14 of the catheter 10 has an overall length 21 that may range from approximately 20-40 inches.

FIG. 3D is an end view of the distal bend 22 depicting the radius of curvature 62 for the bend 22. As shown therein, the radius of curvature 62 may range from approximately 0.25-0.75 inches, and in one particular embodiment it may be approximately 0.50 inches. Moreover, the distal bend 22 may have an included angle 64 that, in one illustrative embodiment, ranges from approximately 75°-150°, in another embodiment ranges from approximately 90°-120°, and in one particular embodiment is approximately 100°.

As depicted in the drawings, the centerline 58 of the distal section 23 is oriented at a compound angle with respect to the plane 29 (see FIG. 3B) containing the proximal section 16, the proximal bend 18 and the intermediate section 20. That is, the centerline of the distal section 23 is oriented at a vertical angle 54 (see FIG. 3B) with respect to the plane 29 and at a horizontal angle 60 (see FIG. 3C) with respect to the plane 29. Thus, the plane 59 (see FIG. 3C) containing the distal bend 22 is oriented at least one angle with respect to the plane 29 containing the proximal section 16, the proximal bend 18 and the intermediate section 20. Through the unique combination of bends 18 and 20, the catheter 10 of the present invention may be used to improve the placement of a lead in a cardiac vein of a patient.

The catheter 10 of the present invention may be introduced into a patient by a variety of known techniques. For example, in one technique, a combined needle and sheath structure (not shown) are used to locate the desired entry point vein. When the proper vein has been located, a syringe is used to draw blood to confirm that the desired vein has been perforated. Thereafter, the sheath is extended further into the vein, and the needle is withdrawn. The catheter 10 may also be introduced with a standard introducer using the Seldinger technique. Then, a relatively soft strengthening member (not shown) may be introduced into the catheter 10 to straighten the catheter 10 and to provide some additional rigidity to the catheter 10 without making it so stiff that it cannot be guided around the coronary sinus. For example, a relatively soft wire, a diagnostic catheter, an obdurator, a steerable catheter, etc., may be used as the straightening member. The catheter shaft 14, along with the straightening member positioned therein, is then routed through the sheath into the vein and guided to the desired area of the heart, e.g., the right atrium. In some embodiments, the strengthening member may not be used or required.

Thereafter, the surgeon may manipulate the catheter 10 so as to position the distal opening 24 and distal bend 22 into the coronary sinus. Once the catheter 10 is positioned in the coronary sinus, the following technique may be employed to position the distal opening 24 in the desired cardiac vein. Initially, the catheter 10 with the inserted strengthening member may be fully inserted to the end of the coronary sinus. Then, the straightening member may be removed partially or totally. Thereafter, the surgeon may slowly retract the distal opening 24 of the catheter 10 down the coronary sinus while continuously introducing a very small volume of a fluoroscopic dye. As the distal opening 24 of the catheter 10 passes over the opening of a cardiac vein, the dye is directed to the opening of the vein, thereby allowing ready identification of the location of the cardiac vein. Additionally, as the distal opening 24 of the catheter 10 passes over a cardiac vein opening, it may deflect into the opening of the cardiac vein due to the presence of the distal bend 22. Once the surgeon directs the catheter 10 to the desired cardiac vein, the catheter 10 may be urged slightly inward so as to position the distal opening 24 more fully into the desired cardiac vein. Then, a lead, such as a pacing or defibrillation lead, may be inserted through the lumen 28 in the catheter 10 and urged forward towards the distal opening 24 until such time as the distal end of the lead exits the distal opening 24 of the catheter 10 and is extended further into the selected cardiac vein. Once the lead is properly positioned and secured within the vein, the catheter 10 may be removed from the patient, leaving the lead behind in the cardiac vein. In one embodiment, the catheter may be slit to remove it from the patient.

The catheter 10 of the present invention provides several advantages over prior art catheters and methods. As an initial matter, the catheter 10 reduces the forces on a lead subsequently introduced into a cardiac vein in the heart. That is, due to the presence of the distal section 23 of the catheter in the cardiac vein, the lead does not experience as much frictional and impingement forces as compared to a lead being implanted without benefit of the catheter 10 to guide the lead into the catheter vein. Reducing the friction allows the surgeon to more easily steer and push the lead into the cardiac vein. Simply put, the present invention removes the need to steer the lead around the corner into the cardiac vein. Given the relatively flexible nature of stylet/lead combinations, this is very important as it may save a surgeon's time and effort and reduce the duration of the implantation operation on the patient. Moreover, through use of the present invention, the amount of fluoroscopic dye that may be employed in an implantation procedure may be reduced.

The present invention is generally directed to a uniquely shaped catheter comprised of multiple bends. In one illustrative embodiment, the catheter 10 comprises a hub 12, a shaft 14 comprised of a lumen 28 that is adapted to have a lead positioned therein, a proximal section 16 coupled to the hub 12, an intermediate section 20, a distal section 23, and a distal opening 24, a proximal bend 18 in a first plane, the proximal bend 18 connecting the proximal section 16 and the intermediate section 20, a distal bend 22 in a second plane, the distal bend 22 connecting the intermediate section 20 and the distal section 23. In further embodiments, the first and second planes may be positioned at least one angle with respect to one another.

In yet another embodiment, the catheter comprises a hub 12, a shaft 14 comprised of a proximal section 16, an intermediate section 20, a distal section 23, a proximal bend 18 connecting the proximal section 16 and the intermediate section 20, and a distal bend 22 connecting the intermediate section 20 and the distal section 23. In this example, the proximal section 16, the proximal bend 18, and the intermediate section 20 are all positioned within a first plane 29, and the distal section 23 has a centerline 58 that is oriented at a compound angle with respect to the first plane 29 as defined by a vertical angle 54, and a horizontal angle 60 between the centerline 58 of the distal section 23 and the first plane 29.

The particular embodiments disclosed above are illustrative only, as the invention may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

1. A catheter for introducing a lead into a heart, comprising:
a hub;
a shaft, forming a lumen that is adapted for positioning a lead therein, the shaft extending from a proximal section coupled to the hub to a distal section including a distal end having a distal opening formed thereon, and having an intermediate section positioned between the proximal section and the distal section;
a proximal bend formed along the shaft in a first plane, the proximal bend positioned between the proximal section and the intermediate section; and
a distal bend formed along the shaft in a second plane, the distal bend positioned between the intermediate section and the distal section.

2. The catheter of claim 1, wherein the second plane is positioned at an angle relative to the first plane.

3. The catheter of claim 1, wherein the proximal section and the intermediate section are positioned at a first angle in the first plane with respect to one another, the first angle ranging from approximately 90-165 degrees.

4. The catheter of claim 1, wherein the intermediate section and the distal section are positioned at a second angle in the second plane with respect to one another, the second angle ranging from approximately 75-150 degrees.

5. The catheter of claim 1, wherein the proximal bend has a radius of curvature of approximately 0.5-1.0 inches.

6. The catheter of claim 1, wherein the distal bend has a radius of curvature of approximately 0.25-0.75 inches.

7. The catheter of claim 1, wherein the proximal section has an axial length of approximately 20-40 inches.

8. The catheter of claim 1, wherein the intermediate section has an axial length of approximately 0.5-3.0 inches.

9. The catheter of claim 1, wherein the distal section has an axial length of approximately 0.5-1.0 inches.

10. The catheter of claim 1, further comprising a tip having a hardness less than 40 durometer positioned adjacent the distal opening.

11. The catheter of claim 1, wherein a centerline of the distal section is disposed at a compound angle with respect to the first plane.

12. The catheter of claim 1, wherein a centerline of the distal section is disposed at a vertical angle of approximately 85-105° and at a horizontal angle of approximately 90-150° with respect to the first plane.

13. The catheter of claim 1, wherein the shaft has an outside diameter that ranges from approximately 4-10 French.

14. The catheter of claim 1, wherein the shaft is formed of a polymer material.

15. The catheter of claim 1, wherein the proximal section and the intermediate section are positioned at a first angle in the first plane with respect to one another, the first angle being approximately 135 degrees.

16. The catheter of claim 1, wherein the intermediate section and the distal section are positioned at a second angle in the second plane with respect to one another, the second angle being approximately 100 degrees.

17. The catheter of claim 1, wherein the lumen is adapted to receive at least one of a pacing lead and a defibrillator lead.

18. The catheter of claim 1, wherein the distal section of the catheter is adapted to be positioned in at least one of a great cardiac vein, a middle cardiac vein, a posterial lateral cardiac vein, an anterial lateral cardiac vein, a right ventricle and a right atrium.

19. A catheter for introducing a lead into a heart, comprising:
a hub; and
a shaft, forming a lumen adapted for positioning of the lead therein, the shaft extending from a proximal section coupled to the hub to a distal section and having an intermediate section positioned between the proximal section and the distal section, a proximal bend positioned between the proximal section and the intermediate section, and a distal bend positioned between the intermediate section and the distal section, the proximal section, the proximal bend, and the intermediate section all positioned within a first plane, the distal section having a centerline that is oriented at a compound angle with respect to the first plane as defined by a vertical angle and a horizontal angle between the centerline of the distal section and the first plane.

20. The catheter of claim 19, wherein the vertical angle ranges from approximately 85-105 degrees.

21. The catheter of claim 20, wherein the horizontal angle ranges from approximately 90-150 degrees.

22. The catheter of claim 19, wherein the proximal bend has a radius of curvature of approximately 0.5-1.0 inches.

23. The catheter of claim 19, wherein the distal bend has a radius of curvature of approximately 0.25-0.75 inches.

24. The catheter of claim 19, wherein the proximal section has an axial length of approximately 20-40 inches.

25. The catheter of claim 19, wherein the intermediate section has an axial length of approximately 0.5-3.0 inches.

26. The catheter of claim 19, wherein the distal section has an axial length of approximately 0.5-1.0 inches.

27. The catheter of claim 19, further comprising a tip having a hardness less than 40 durometer positioned adjacent the distal opening.

28. The catheter of claim 19, wherein the shaft has an outside diameter that ranges from approximately 4-10 French.

29. The catheter of claim 19, wherein the shaft is formed of a polymer material.

30. The catheter of claim 19, wherein the lumen is adapted to receive at least one of a pacing lead and a defibrillator lead.

31. The catheter of claim 19, wherein the distal section of the catheter is adapted to be positioned in at least one of a great cardiac vein, a middle cardiac vein, a posterial lateral cardiac vein, an anterial lateral cardiac vein, a right ventricle and a right atrium.

32. A catheter for introducing a lead within a heart, comprising:
a hub;
a shaft, forming a lumen that is adapted for positioning a lead therein, the shaft extending from a proximal section coupled to the hub to a distal section including a distal end having a distal opening formed thereon, and having an intermediate section positioned between the proximal section and the distal section;
a proximal bend formed along the shaft in a first plane, the proximal bend positioned between the proximal section and the intermediate section; and
a distal bend formed along the shaft in a second plane, the distal bend positioned between the intermediate section and the distal section, wherein the second plane is positioned at an angle relative to the first plane, the proximal section and the intermediate section are positioned at a first angle in the first plane with respect to one another, the first angle ranging from approximately 90-165 degrees, the intermediate section and the distal section are positioned at a second angle in the second plane with respect to one another, the second angle ranging from approximately 75-150 degrees, the proximal bend has a radius of curvature of approximately 0.5-1.0 inches, the distal bend has a radius of curvature of approximately 0.25-0.75 inches, the proximal section has an axial length of approximately 20-40 inches, the intermediate section has an axial length of approximately 0.5-3.0 inches, and the distal section has an axial length of approximately 0.5-1.0 inches.
